(19) United States
(12) Patent Application Publication
(10) Pub. No.: US 2026/0207808 A1
(43) Pub. Date: Jul. 23, 2026

(54) DEVICE FOR HANDLING STERILE-MATERIAL CONTAINER SYSTEMS

(71) Applicant: Aesculap AG, Tuttlingen (DE)

(72) Inventors: Arne Jansen-Troy, Stockach (DE); Allan Maas, Konstanz (DE); Daniel Kiessling, Villingen-Schwenningen (DE); Matthias Henke, Villingen-Schwenningen (DE); Stephan Dorau, Herzogenaurach (DE); Dennis Goerz, Tuttlingen (DE)

(21) Appl. No.: 19/118,880

(22) PCT Filed: Oct. 6, 2023
(86) PCT No.: PCT/EP2023/077749

§ 371 Date: Apr. 7, 2025

(30) Foreign Application Priority Data
Oct. 7, 2022 (DE) 10 2022 125 967.6

(51) Int. Cl. A61L 2/26; A61B 50/30; B25J 9/16

(52) U.S. Cl. CPC A61L 2/26; A61B 50/30; B25J 9/1687

(57) ABSTRACT

A method for handling sterile-material container systems, in particular for separating sterile-material container systems, includes the steps of removing sterile-material containers located in a container transport cart one after the other, and opening the containers. A filter holder is removed from a container lid of each sterile-material container. Used filter inserts arranged in the filter holder are removed. The emptied filter holder is replaced back on the container lid, and the container lid is placed on a conveyor belt. A strainer basket with used medical instruments is removed from a container tub of the sterile goods container and placed on the conveyor belt. These steps, which can be fully-automatic or semi-automatic, are repeated until the container transport cart is completely empty. The emptied container transport cart is then transported to a cleaning station. The method can be carried out by a sterile-material container handling device.

### CROSS-REFERENCE TO RELATED APPLICATIONS

This application is the United States national stage entry of International Application No. PCT/EP 2023/077749, filed on Oct. 6, 2023, and claims priority to German Application No. 10 2022 125 967.6, filed on Oct. 7, 2022. The contents of International Application No. PCT/EP 2023/077749 and German Application No. 10 2022 125 967.6 are incorporated by reference herein in their entireties.

### FIELD

The present disclosure relates to a method for handling sterile-material container systems and to a device for performing the method for handling sterile-material container systems.

### BACKGROUND

In medical care, ever-increasing demands are being put on efficiency with high quality and hygiene requirements at the same time. This particularly applies to processes involved in the handling of medical devices. These processes include, for example, cleaning and sterilization, storage and transport processes, as well as processes in the area of a reprocessing unit for medical devices (AEMP))/Central Sterile Supply Department (CSSD)/Sterile Processing Department (SPD). In particular, in separating (disassembling) and assembling structural and functional groups of sterile-material container systems and possibly their contents, which are each part of the AEMP, it is of essential importance to prevent handling errors in light of patient safety. In other words, processes are developed for handling medical devices including their storage and sterilization containers, which must be executed accurately and without errors in order to comply with existing hygiene regulations. However, relieving employees of heavy, non-value-adding work such as lifting as well as loading and unloading sterile-material containers is also becoming increasingly important. In order to prevent errors in the handling of medical products and work equipment, such as the separation and/or assembly of components and functional groups of sterile-material container systems, while at the same time increasing efficiency and likewise reducing the workload on workers, these processes can be automated and carried out by robots.

In the prior art, automated/robotic processes are known that describe the handling of individual components and functional groups of sterile-material container systems, which focuses on individual logistical tasks such as transporting and loading and/or unloading sterile-material containers. This reduces automated handling processes to just a few process sequences. A major problem with further process sequences, such as dismantling medical systems into their components and/or functional groups and then transferring components/assemblies and functional groups to subsequent cleaning and/or sterilization stations, is that components/assemblies and functional groups, for example of a sterile goods container system, are each designed with different components of an electronic or mechanical nature, which consequently require different handling. It is also possible that several components and functional groups are equal with regard to their handling and therefore can be handled in the same way. Thus, a kind of selection of the respective component/functional groups is required with regard to the respective subsequent handling, which should always be as economical and fast as possible. This in turn requires a specific process flow depending on the component and/or functional groups to be expected.

### SUMMARY

One purpose of the present disclosure is to provide a method for handling sterile-material container systems that enables automated, error-free and more efficient handling, for example using robotic effectors/manipulators.

This task is solved by a method of the present disclosure, as well as a system arrangement.

Specifically, the task is solved by a method for handling sterile-material containers, which has at least the following steps:
First, a sterile-material container is removed from the container transport cart using a first manipulator. It is particularly preferably to empty the container transport cart using an algorithm such that sterile-material containers at the top are taken and respectively unloaded first and then the sterile-material containers underneath. Alternatively, a user can define a prioritization and/or an emptying sequence via a user interface, so that with the aid of previous scanning the desired sterile-material container can be detected and grasped. Based on the algorithm used, the sterile-material container located on the desired sterile-material container is unloaded first. If, for example, the prioritized sterile-material container is in second row and possibly is not directly visible, the first row of sterile-material containers is systematically unloaded until the corresponding prioritized sterile-material container is recognized (clearance mode). After recognition, it is unloaded according to its position.
In a next step, the removed sterile-material container is opened in that a second manipulator is approached by the first manipulator. Alternatively, both manipulators can move to a point or area in the respective work area that is appropriate for them. The second manipulator preferably has an opening and holding mechanism/effector for opening and holding a container lid and opens/removes the container lid by means of a translational relative motion of the tools/effectors/mechanisms of the first and second manipulators.
In the next step, a filter holder of the sterile-material container, which is usually located on the container lid, is emptied. The filter holder is emptied in that a third manipulator is approached by the second manipulator holding the container lid and the third manipulator actuates an opening mechanism of the filter holder. By means of a translational relative motion, the filter holder can now be removed from the container lid, for example, by way of the third manipulator. However, emptying of the filter holder is preferably carried out with the aid of a further (fourth) manipulator designed for this purpose and/or a blast of compressed air and/or a suction device.

Used/spent filters are removed from the filter holder and collected in a storage container.
After emptying the filter holder, the filter holder and the container lid, in a next step, are reassembled by way of a translational relative motion of the second and third manipulators to each other, so that the filter holder and the container lid are reconnected to each other to form a functional unit.
In the next step, the assembled container lid including the filter holder is put in a shelving system or on a means of transport with the help of the second manipulator. The preferred means of transport is a conveyor belt, particularly preferably executed as a FIFO conveyor, which automatically transports the container lid for further handling, such as cleaning.
In the subsequent step, the second manipulator moves towards the first manipulator, which continues to hold a container tub of the sterile-material container. The second manipulator moves its tools/effectors/mechanisms so that it can grasp/pick up a strainer basket located inside the container tub. Grasping/picking up can be effected at handles and/or anchor points of the handles of the strainer basket. With a translational relative motion of the first and second manipulators to each other, the strainer basket is removed from the container tub by the second manipulator and delivered to yet another shelving system or to a further means of transport. Those further means of transport transfer the functional groups stored on/in them to respective further handling/processing stations. Thus, the removal and separation of a single sterile-material container into its functional groups and/or components, as well as their respective transfer to the corresponding subsequent processing stations is completed.

The described steps now are repeated for each individual sterile-material container in the container transport cart until it is completely emptied or the predefined desired number of sterile-material containers was removed.

According to the invention, the described method steps are fully automated. It should be noted at this point that a sterile-material container can be assembled in the same way using the same method steps, but in reverse order and orientation.

Preferably, the method step of removing the sterile-material container from the container cart is preceded by the step of transporting the container transport cart with the sterile-material containers therein to a previous handling station. The transport of the container transport cart can preferably be carried out with the aid of a conveyor belt, a so-called Automated Guided Vehicle (AGV) or, particularly preferably, self-propelled.

In a further step, which takes place immediately after transporting the container transport cart to the handling station, the doors of the container transport cart are opened by a catch. Preferably, the doors of the container cart are designed as sliding doors or roller shutters or as hinged doors.

In the subsequent step, tags and/or markers of the opened container transport cart are tracked. Tracking is preferably carried out using optical and/or ultrasonic sensors and/or radio. Furthermore, the tags and/or markers are assigned to the sterile containers. More specifically, a position of the sterile-material containers within the container transport cart is assigned through tracking.

In a last step, which takes place immediately after transfer of the strainer basket and the container tub to the shelving system or to the means of transport, the emptied container transport cart is transported to cleaning. The container transport cart can be transported preferably by way of a conveyor belt, a so-called Automated Guided Vehicle (AGV) or, even more preferably, self-propelled.

Preferably, the step of removing a sterile-material container from the container transport cart is controlled by means of an algorithm.

The task of the present disclosure moreover is solved by a sterile-material container handling device for carrying out a handling process, in particular according to the described process for handling sterile-material container systems.

The sterile-material container handling device comprises at least one control unit which is designed such that the handling process is controlled automatically and in which a machine-readable data carrier is or can be provided, on which the previously described method is stored in the form of a corresponding program. Furthermore, the sterile-material container handling device includes a container transport cart for transporting sterile-material containers located therein to and/or from a handling station. Moreover, the sterile-material container handling device has a robotic system including at least one or more manipulator(s). The at least one manipulator is controlled by the control unit such that the following handling steps are carried out:
Individually removing and/or transporting sterile-material containers from and/or to the container transport cart
Disassembling and/or assembling the sterile-material container into or from individual container components, as well as
Removing and/or returning strainer baskets, which preferably contain medical work equipment, from or to the sterile-material container.

Furthermore, the sterile-material container handling device preferably includes a monitoring device for tracking tags and/or markers in the opened container transport cart and assigning the tags and/or markers to the sterile-material containers and their coordinates. In addition, the sterile-material container handling device preferably includes means of transport for transporting strainer baskets separately from the container components to or from the handling station.

Preferably, the at least one manipulator has mechanisms/effectors/tools that are designed to open, hold and close the sterile-material containers and preferably also to remove sterile-material baskets.

Preferably, the system arrangement can both separate sterile-material container systems according to the method described above and join them together.

For reassembly of the sterile-material container systems, i.e. systems already cleaned, the steps of the method for handling sterile-material container systems are carried out in reverse order, as was already indicated above, the components needing to be supplied to the manipulators in a defined manner and in the respective reverse direction as described above with regard to disassembly. This applies in particular to the filters which must be inserted into the filter holder by another manipulator.

### BRIEF DESCRIPTION OF THE DRAWING FIGURES

FIG. 1 shows a schematic representation of the sequence of process steps according to the present disclosure; and

FIG. 2 shows a representation of the sterile-material container handling device according to the present disclosure.

### DETAILED DESCRIPTION

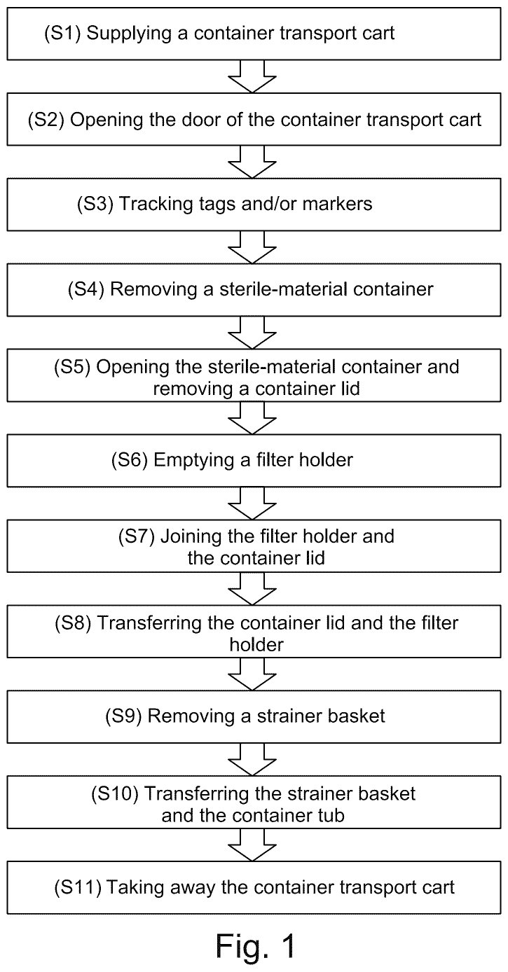

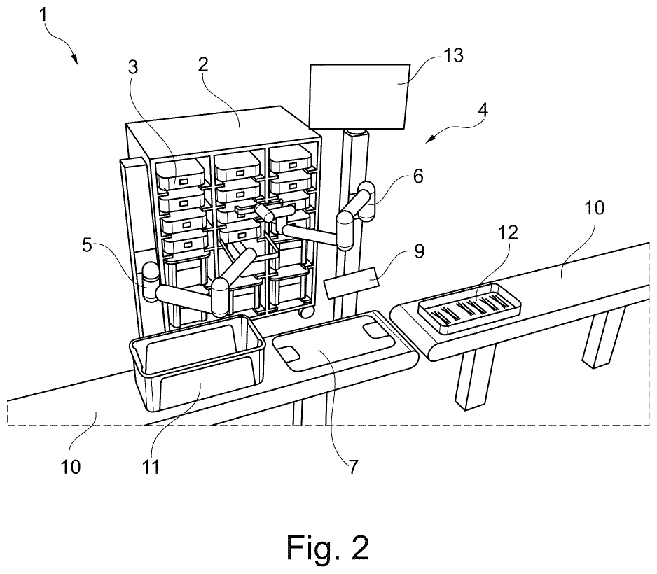

FIG. 1 shows a schematic sequence of the method steps of the method for handling sterile-material container systems according to the present disclosure. Preferably, the embodiment for unloading a container transport cart 2 is described. The method is carried out with reference to the sterile-material container handling device 1 shown in FIG. 2. Accordingly, method steps S1 to S11 are processed serially and implemented in practice by way of the associated manipulators according to the subsequent description. However, it is also possible that individual method steps be carried out in parallel, in particular, if different manipulators are used for this purpose. This is explained in more detail subsequently.

The sterile-material container handling device 1 comprises, according to FIG. 2, a container transport cart 2 in which a plurality of sterile-material containers 3 is arranged. The sterile-material containers 3 are stacked on top of each other inside the container transport cart 2 and are arranged in several rows. Each sterile-material container 3 moreover has a container lid 7 and a container tub 11, which together form the sterile-material container 3.

Furthermore, the present sterile-material container handling device 1 has at least three manipulators 5, 6, 9 which can be controlled separately by a control unit 13 (in the present case, an input-output interface in the form of a monitor or touch screen with an integrated computing unit is shown) and are preferably formed as robot arms, but can also take other forms such as conveyor belts, sliders, etc. The manipulators 5, 6, 9 are arranged in relation to one another such that their operating ranges overlap at least for two manipulators, respectively, so that the manipulators in question interact. Moreover, the position of the manipulators in relation to the depicted container transport cart 2 is defined such that the cart likewise is located in the operating range of at least one or two manipulators.

The sterile-material container handling device 1 is part of a handling station 4, which also includes two conveyor belts 10 that run separately from each other and are preferably aligned in different directions to each other and lead to different further handling stations. At least one end section of each of the two conveyor belts 10 lies in the operating range of at least one of the manipulators, with each end section serving as a support or removal area for assemblies/functional groups of the respective sterile-material container system currently handled.

In summary, the handling station 4 for handling the sterile-material container systems is structured so that, starting from an operating or service person (who is looking at the touch screen or monitor 13 of the control unit), the container transport cart 2, is located behind the sterile-material container handling device 1, and the conveyor belts 10 are arranged in front of the sterile-material container handling device 1. In this way, the container transport cart 2 and the conveyor belts 10 physically limit the effective ranges of manipulators 5, 6, 9 and thus serve as protective barriers for the operating/service person.

Finally, the manipulators 5, 6, 9 are mounted on at least two columns spaced along the conveyor belts 10, of which at least one also serves as a holder for the touch screen or monitor 13 of the control unit, which thus towers above all components of the handling station 4.

The mode of operation of the handling station 4 can be described as follows:

In a first step S1, the container transport cart is transported to a handling station 4, the sterile-material container handling device 1 of which has the three manipulators 5, 6, 9, as described above. However, more or less manipulators can be provided for, depending on the effectors mounted on them and their multiple possibly executable functions.

In a next step S2, the door of the container transport cart 2 is opened, which can be done manually or automatically with the help of (first) manipulator 5. In a subsequent third step S3 tags and/or markers or similar labels are tracked by way of a scanner and/or imaging system not shown in further detail, whereby the arrangement and the coordinates of the sterile-material container 3 within the container transport cart 2 are determined, so that with the help of a matching algorithm, unloading of the container transport cart 2 can be controlled and started. The algorithm is stored in the control unit 13 and can be changed/adapted, if necessary, via the input/output interface and the touch screen 13, respectively. This makes it possible to unload the containers from the transport cart in a specific order or to deliberately approach specific containers, in which case previous clearing/providing accessibility possibly has to be carried out.

In a fourth step S4, the first manipulator 5 removes a sterile-material container 3, so that in the next step S5 the sterile-material container 3 is opened with the aid of the second manipulator 6 in that the second manipulator 6 removes the container lid 7 with a translational relative motion. In the subsequent step S6, a third manipulator 9 removes a filter holder 8 (not shown) located on the container lid 7 and empties it with the help of a blast of compressed air or a suction device.

Subsequently, in step S7, the filter holder 8 is put back on the container lid 7 and transferred to the first means of transport 10, which is formed as a conveyor belt (corresponds to step S8). In the following step S9, the second manipulator 6 removes a strainer basket 12 located in the sterile-material container 3, in which medical work equipment is located. In the next step S10, the strainer basket 12, by means of the second manipulator 6, is transferred to the second means of transport 10, which likewise is designed as a conveyor belt. In the same step, the first manipulator 5 transfers the container tub 11 to the first means of transport 10 arranged axially next to the second means of transport. The steps S1-S10 are repeated until the entire container transporter 2 is emptied or the algorithm, which was previously pre-programmed via a user interface 13, has been completed. Subsequently, as a final step S11, the container cart 2 is taken away.

### LIST OF REFERENCE SIGNS

1 Sterile-material container handling device
2 Container transport cart
3 Sterile-material container
4 Handling station
5 Manipulator
6 Manipulator
7 Container lid
8 Filter holder
9 Manipulator
10 Means of transport
11 Container tub
12 Strainer basket
13 User interface

1.-13. (canceled)

14. A method for handling sterile-material container systems comprising at least the following steps:
S4: removing at least one sterile-material container from a container transport cart with an automatic manipulator;
S5: opening the at least one sterile-material container by moving the automatic manipulator or a further automatic manipulator in position and removing a container lid of the at least one sterile-material container;
S6: emptying a filter holder of the at least one sterile-material container by moving the automatic manipulator or a further automatic manipulator in position and opening the filter holder with the automatic manipulator or the further automatic manipulator;
S7: joining the filter holder and the container lid;
S8: transferring the container lid and the filter holder to a shelving system or to a means of transport;
S9: removing a strainer basket located in a container tub of the at least one sterile-material container by approaching the automatic manipulator or a further automatic manipulator;
S10: transferring the strainer basket and the container tub into the shelving system or to the means of transport; and
repeating steps S4-S10 until the container transport cart has been completely emptied,
wherein steps S4-S10 are fully automated.

15. The method according to claim 14, further comprising a step of
S1: automatically delivering the container transport cart with the at least one sterile-material container to a handling station.

16. The method according to claim 15, further comprising a step of
S2: automatically opening a door of the container transport cart immediately after the step of S1: automatically delivering the container transport cart.

17. The method according to claim 16, further comprising the step of
S3: automatically tracking a tag and/or a marker in the container transport cart and assigning the tag and/or the marker to the at least one sterile-material container and coordinates of the at least one sterile-material container, immediately prior to the step of S4: removing at least one sterile-material container from a container transport cart.

18. The method according to claim 17, wherein step S3: automatically tracking a tag and/or a marker in the container transport cart is carried out with optical sensors and/or ultrasonic sensors and/or radio.

19. The method according to claim 17, further comprising the step of
S11: automatically transporting the container transport cart away for cleaning.

20. The method according to claim 19, wherein step S1: automatically delivering the container transport cart and step S11: automatically transporting the container transport cart are executed with a conveyor belt or an automated guided vehicle.

21. The method according to claim 14, wherein step S4: removing at least one sterile-material container from a container transport cart is controlled by an algorithm.

22. The method according to claim 14, wherein step S6: emptying a filter holder of the at least one sterile-material container is carried out with a manipulator and/or a blast of compressed air and/or a suction device.

23. The method according to claim 14, wherein the method is used for separating sterile-material container systems.

24. A machine-readable data carrier of or for a sterile-material container handling device, wherein the machine-readable data carrier stores the method according to claim 14.

25. A sterile-material container handling device configured to carry out the method for handling sterile-material container systems according to claim 14.

26. A sterile-material container handling device for carrying out a handling process, the sterile-material container handling device comprising:
at least one control unit for automatically controlling the handling process;
at least one container transport cart for transferring at least one sterile-material container to and/or away from a handling station;
a robotic system with at least one manipulator controlled by the at least one control unit;
a monitoring device for tracking at least one tag and/or marker in the at least one container transport cart and assigning the at least one tag and/or marker to the at least one sterile-material container and coordinates of the at least one sterile-material container; and
a transport for transporting strainer baskets separately to container components and to or from the handling station,
the robotic system being configured to:
open and/or close the at least one container transport cart and individually remove and/or supply the at least one sterile-material container from and/or into the at least one container transport cart, and
disassemble and/or assemble the at least one sterile-material container into or from individual container components, and remove and/or return at least one strainer basket from or to the at least one sterile-material container.

27. The sterile-material container handling device according to claim 26, further comprising an opening and holding mechanism of the at least one manipulator.

28. The sterile-material container handling device according to claim 26, wherein the sterile-material container handling device carries out both a separation and a joining of sterile-material containers of the sterile-material container handling device.

29. A machine-readable data carrier of or for the sterile-material container handling device according to claim 26.
